(19) United States
(12) Patent Application Publication
(10) Pub. No.: US 2026/0026963 A1
(43) Pub. Date: Jan. 29, 2026

(54) SYSTEMS AND METHODS FOR SELECTIVELY ADJUSTING THE SLEEPING POSITION OF A USER

(71) Applicant: RESMED DIGITAL HEALTH INC., San Diego, CA (US)

(72) Inventors: Michael James Dent, Bella Vista, New South Wales (AU); Sakeena De Souza, Bella Vista, New South Wales (AU); Andrew Chan, Bella Vista, New South Wales (AU); Zhou QI Lee, Singapore (SG); Garth Alan Berriman, Bella Vista, New South Wales (AU); Line Andersen, Bella Vista, New South Wales (AU)

(21) Appl. No.: 18/997,748

(22) PCT Filed: Jul. 22, 2023
(86) PCT No.: PCT/US2023/028423

§ 371 Date: Jan. 22, 2025

Related U.S. Application Data
(60) Provisional application No. 63/369,166, filed on Jul. 22, 2022.

(51) Int. Cl. A61F 5/56; A61G 7/015; A61G 7/018; G16H 40/67

(52) U.S. Cl. CPC A61F 5/56; A61G 7/015; A61G 7/018; G16H 40/67

(57) ABSTRACT

A method includes receiving sleeping data corresponding to an actual sleeping position of a user. The method also includes determining the actual sleeping position on an adjustable underlay. where the adjustable underlay having a plurality of selectively adjustable segments. The method also includes comparing the actual sleeping position to an intended sleeping position of the user. The method also includes adjusting the adjustable underlay to encourage the user to change the actual sleeping position to the intended sleeping position, in response to determining that the actual sleeping position is different than the intended sleeping position.

### CROSS-REFERENCE TO RELATED APPLICATIONS

This application claims the benefit of, and priority to, U.S. Provisional Patent Application No. 63/369,166, filed Jul. 22, 2022, which is hereby incorporated by reference herein in its entirety.

### TECHNICAL FIELD

The present disclosure relates generally to systems and methods for improving quality of sleep of a user, and more particularly, to systems and methods for selectively adjusting the sleeping position of a user using an adjustable underlay.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders usually have a negative impact on the quality of sleep experienced by the individual. Some disorders are often treated using a respiratory therapy system (e.g., a continuous positive airway pressure (CPAP) system), which delivers pressurized air to aid in preventing the individual's airway from narrowing or collapsing during sleep. However, other disorders correspond to the position in which the user sleeps, and therefore are not solved as a result of using a respiratory therapy system.

For instance, individuals often have a difficult time finding and staying in a comfortable sleeping position. According to an example, pregnant women and people with chronic back pain suffer from poor sleep because they have difficulty finding, and staying in, a comfortable sleeping position. Additionally, many people struggle from disabilities, and stiffness, chronic back pain, age, etc., and others prefer sleeping in a certain position that would not be improved by using a respiratory therapy system. The present disclosure is directed to solving these and other problems.

### SUMMARY

According to some implementations of the present disclosure, a method includes receiving sleeping data corresponding to an actual sleeping position of a user. The method also includes determining the actual sleeping position on an adjustable underlay. The adjustable underlay includes a plurality of selectively adjustable segments, each of which may be inflated and/or deflated. The method also includes comparing the actual sleeping position to an intended sleeping position of the user. The method also includes adjusting the adjustable underlay to encourage the user to change the actual sleeping position to the intended sleeping position in response to determining that the actual sleeping position is different than the intended sleeping position.

According to some implementations of the present disclosure, a system includes an adjustable underlay, a memory, and a control system. The adjustable underlay includes a plurality of selectively adjustable segments. The memory stores machine-readable instructions. The control system includes one or more processors configured to execute the machine-readable instructions to receive sleeping data corresponding to an actual sleeping position of a user. The control system is further configured to determine the actual sleeping position on an adjustable underlay, the adjustable underlay having a plurality of selectively adjustable segments. The control system is further configured to compare the actual sleeping position to an intended sleeping position of the user. The control system is further configured to adjust the adjustable underlay to encourage the user to change the actual sleeping position to the intended sleeping position in response to determining that the actual sleeping position is different than the intended sleeping position.

According to some other further implementations of the present disclosure, a system includes an adjustable underlay, a respiratory therapy system, a memory, and a control system.

The adjustable underlay includes a plurality of selectively adjustable segments, each of which may selectively be inflated or deflated to adjust the amount of air included in the respective segment. The respiratory therapy system is configured to selectively supply pressurized air to a user and/or the adjustable underlay. The memory stores machine-readable instructions. The control system includes one or more processors configured to execute the machine-readable instructions to receive sleeping data corresponding to an actual sleeping position of the user. The control system is further configured to determine the actual sleeping position on an adjustable underlay, the adjustable underlay having a plurality of selectively adjustable segments. The control system is further configured to compare the actual sleeping position to an intended sleeping position of the user. The control system is further configured to adjust the adjustable underlay to encourage the user to change the actual sleeping position to the intended sleeping position in response to determining that the actual sleeping position is different than the intended sleeping position.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure.

FIG. 2A is a perspective view of at least a portion of the system of FIG. 1, a user, and a bed partner, according to some implementations of the present disclosure.

FIG. 2B is a perspective view of a respiratory therapy device of the system of FIG. 1, according to some implementations of the present disclosure.

FIG. 2C is a perspective view of the respiratory therapy device of FIG. 2A illustrating an interior of a housing, according to some implementations of the present disclosure.

FIG. 3A is a representational view of at least a portion of the system of FIG. 2A, a network, and remote locations, according to some implementations of the present disclosure.

FIG. 3B is a cross-sectional view of a portion of the selectively adjustable underlay of FIG. 3A, according to some implementations of the present disclosure.

FIG. 3C is a cross-sectional view of a portion of the selectively adjustable underlay of FIG. 3A, according to some implementations of the present disclosure.

FIG. 4 illustrates an exemplary timeline for a sleep session, according to some implementations of the present disclosure.

FIG. 5 illustrates an exemplary hypnogram associated with the sleep session of FIG. 4, according to some implementations of the present disclosure.

FIG. 6A is a process flow diagram for a method for improving the quality of sleep experienced by a user by selectively adjusting the sleeping position of the user according to some implementations of the present disclosure.

FIG. 6B is a process flow diagram for monitoring the adjustments made to an underlay according to some implementations of the present disclosure.

FIG. 6C is a process flow diagram for a method for improving the quality of sleep experienced by a user by selectively adjusting the sleeping position of the user according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive deoxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

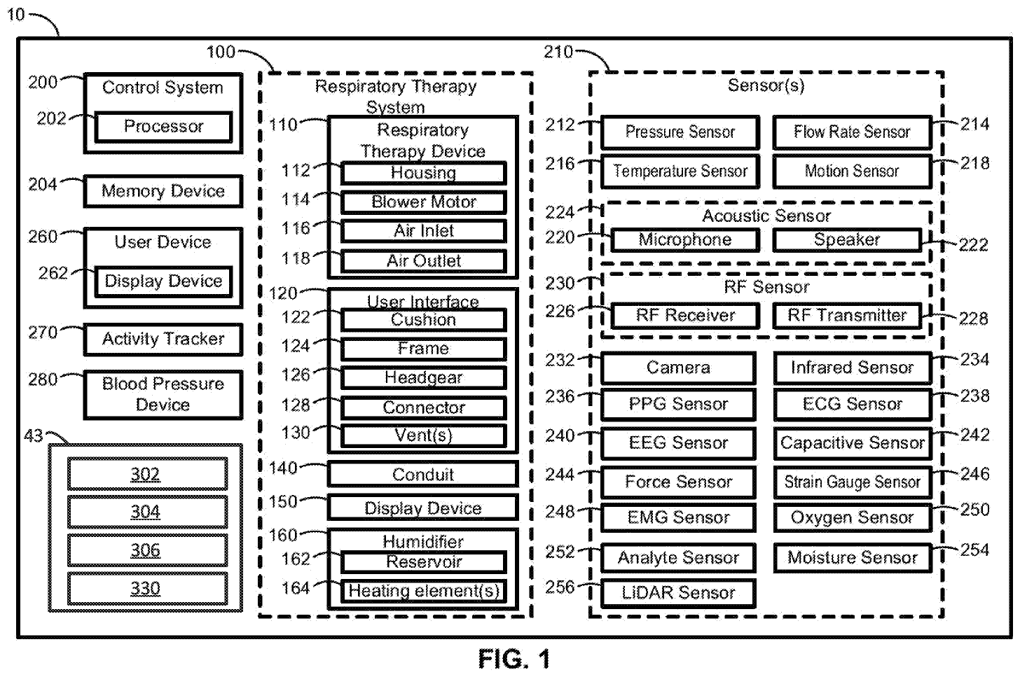

Referring to FIG. 1, a system 10, according to some implementations of the present disclosure, is illustrated. The system 10 includes a respiratory therapy system 100, a control system 200, one or more sensors 210, a user device 260, and an activity tracker 270.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

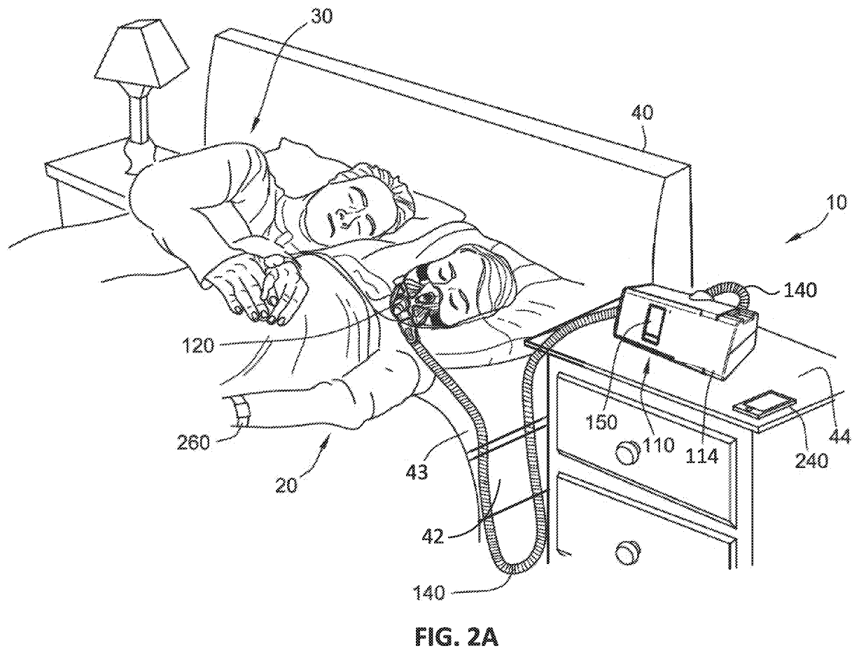

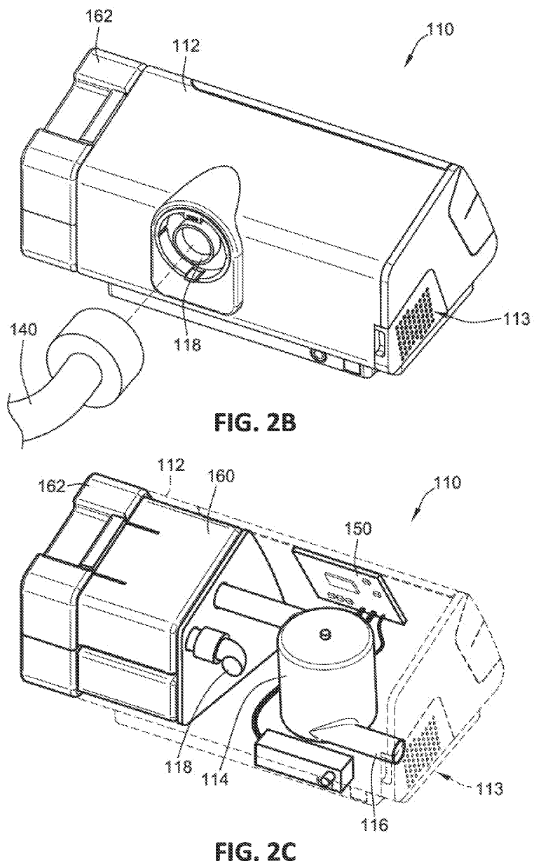

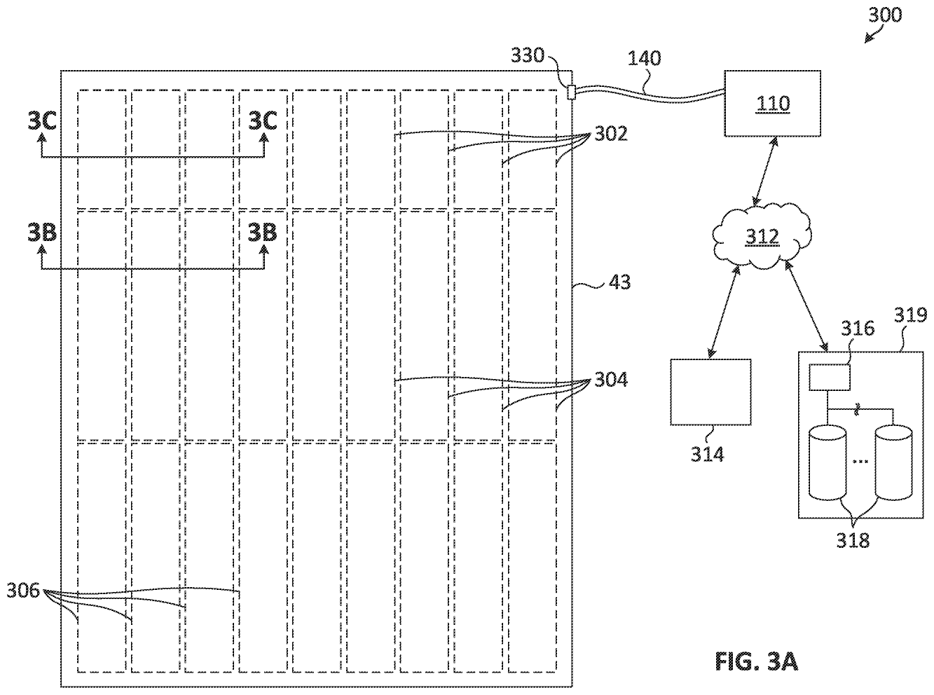

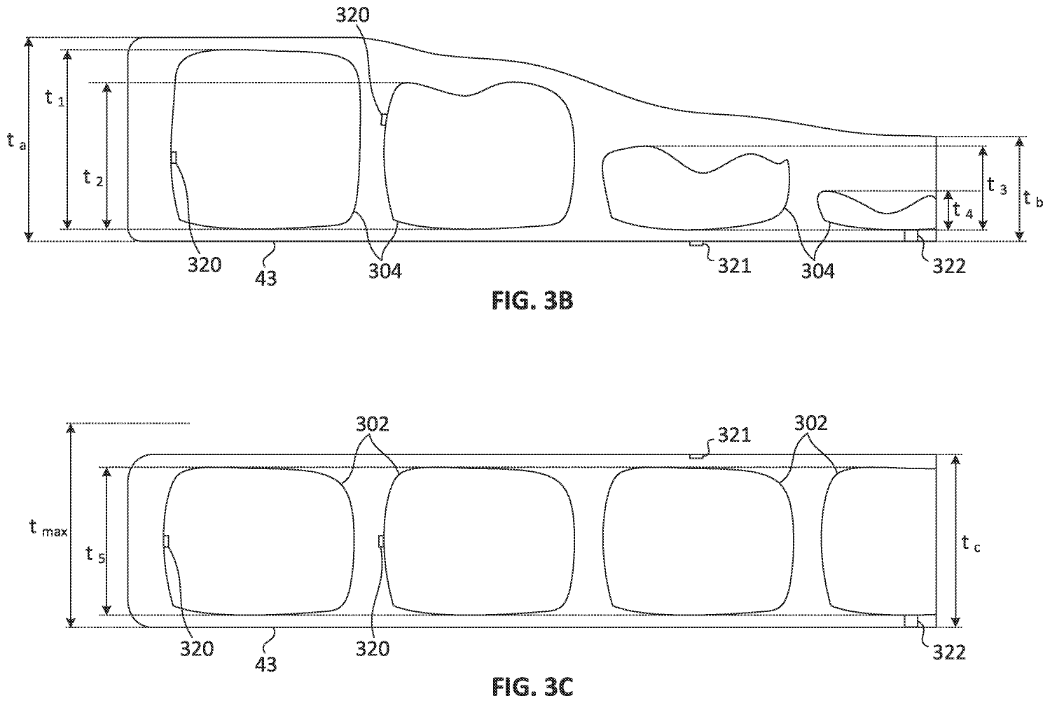

As shown in FIG. 2A, the respiratory therapy system 100 can be used to treat user 20 in combination with a selectively adjustable underlay 43. In this example, the user 20 of the respiratory therapy system 100 and a bed partner 30 are located in a bed 40 and are laying on an adjustable underlay 43 which is positioned above (e.g., on top of) a mattress 42 in the present implementation. The adjustable underlay 43 includes a plurality of selectively adjustable segments (e.g., see FIGS. 3A-3C), each of which may be inflated and/or deflated to achieve a desired configuration of the adjustable underlay 43. The various segments may selectively be inflated and/or deflated in order to support various parts of a user's body while sleeping. Moreover, the selectively adjustable segments may be inflated and/or deflated as desired without affecting properties of the remaining ones of the selectively adjustable segments in the adjustable underlay 43.

According to some implementations, the respiratory therapy device 110 may be used to create the positive and negative pressures associated with inflating and deflating the various segments in the adjustable underlay 43. For example, a blower motor (e.g., see 114 of FIG. 1) in the respiratory therapy device 110 may be used to create the air pressures associated with inflating and deflating the various segments to their respective levels. However, it should be noted that the segments in the adjustable underlay 43 may be inflated and/or deflated using one or more of gasses (e.g., ambient air), liquids (e.g., water), other materials, etc., that would be apparent to one skilled in the art after reading the present description.

With continued reference to FIG. 2A, the respiratory therapy device 110 can be positioned on a nightstand 44 that is directly adjacent to the bed 40 as shown in FIG. 2A, or more generally, on any surface or structure that is generally adjacent to the bed 40 and/or the user 20. Accordingly, the conduit 140 may be used to couple the respiratory therapy device 110 to an inlet (e.g., see 330 of FIG. 3A) of the adjustable underlay 43 to adjust fill levels of the various segments. In response to modifying the adjustable underlay 43 to a desired sleep setting, the conduit 140 may be decoupled from the inlet of the adjustable underlay 43 and coupled to a respiratory therapy device 110. As a result, the respiratory therapy device 110 may be used to fill the various segments in the adjustable underlay 43, as well as generate pressurized air that is delivered to a user. In some embodiments, a second conduit may be provided so that the respiratory therapy device 110 is coupled to both the adjustable underlay 43 and the user interface 120. This will enable respiratory therapy to be provided throughout the night as well as provide the option for the underlay 43 to be adjusted throughout the night when necessary. In further embodiments, the exhaled gases or controlled leak from the user interface 120 may be used to inflate the underlay 43, for example by way of a conduit.

In other implementations, the adjustable underlay 43 may include or be coupled to a dedicated component configured to inflate and/or deflate the various segments. For example, the adjustable underlay 43 may include a motor dedicated to adjusting the amount of air included in each of the various segments. Accordingly, the respiratory therapy device 110 may be dedicated to generating pressurized air that is delivered to a user in some implementations.

It follows that the respiratory therapy device 110 may generally be used to generate pressurized air that is delivered to a user (e.g., using one or more motors that drive one or more compressors) and/or used to adjust the size and firmness of the various segments in the adjustable underlay 43. In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about −20 L/min and about 150 L/min, while maintaining a positive pressure (relative to the ambient pressure).

The respiratory therapy device 110 includes a housing 112, a blower motor 114, an air inlet 116, and an air outlet 118 (FIG. 1). Referring momentarily to FIGS. 2B and 2C, the blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some situations where the respiratory therapy device 110 is used to inflate and/or deflate the various segments in the adjustable underlay 43, the humidifier 160 may selectively be deactivated (e.g., turned off) to reduce run-time and power consumption. In other implementations, the air inlet 116 and/or the air outlet 118 include a cover that is moveable between a closed position and an open position (e.g., to prevent or inhibit air from flowing through the air inlet 116 or the air outlet 118). As shown in FIGS. 2B and 2C, the housing 112 can include a vent 113 to allow air to pass through the housing 112 to the air inlet 116. As described below, the conduit 140 is coupled to the air outlet 118 of the respiratory therapy device 110.

Referring back to FIG. 1, the user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H2O relative to ambient pressure. For other forms of therapy, such as the delivery of oxygen, the user interface may not include a seal sufficient to facilitate delivery to the airways of a supply of gas at a positive pressure of about 10 cmH2O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

In some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

The conduit 140 (also referred to as an air circuit or tube) allows the flow of air between components of the respiratory therapy system 100, such as between the respiratory therapy device 110 and the user interface 120 and/or between the respiratory therapy device 110 and the adjustable underlay 43. In some implementations, there can be separate limbs of the conduit for inhalation and exhalation. In other implementations, a single limb conduit is used for both inhalation and exhalation.

In still other implementations, a first limb of a multiple limb conduit is used to connect the respiratory therapy device 110 and the user interface 120, while a second limb of the multiple limb conduit is used to connect the respiratory therapy device 110 and the adjustable underlay 43. Accordingly, the respiratory therapy device 110 may include at least a second motor in some implementations such that pressurized air may be provided to the user interface 120 simultaneously and in parallel to inflating or deflating the selectively adjustable segments of the adjustable underlay 43. In such implementations, the control system 200 is able to perform various processes associated with providing pressurized air to the user interface 120 simultaneously and in parallel to perform various processes associated with inflating or deflating the selectively adjustable segments of the adjustable underlay 43. In some implementations, the control system 200 is able to make adjustments to the pressurized air provided to the user interface 120 based on the settings (e.g., fill levels) of the segments in the adjustable underlay 43, and vice versa. Thus, the adjustable underlay 43 may operate in unison with the air provided to the user interface 120 such that the user may have an improved quality of sleep, e.g., as would be appreciated by one skilled in the art after reading the present description.

Referring to FIG. 2B, the conduit 140 includes a first end 142 that is coupled to the air outlet 118 of the respiratory therapy device 110. The first end 142 can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end 142 can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

In some implementations, the display device 150 may be used to display image(s) including still images, video images, or both and/or information regarding the adjustable underlay 43. For example, the display device 150 can provide information regarding the status of the segments in the adjustable underlay 43 (e.g., fill level, pressure, etc.). The display device 150 may also serve as a GUI and be configured to receive input from a user that is used to control the segments in the adjustable underlay 43, e.g., to produce a desired sleeping surface to achieve a desired sleeping position.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, as shown in FIGS. 2B-2C, air flow from the air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. As yet another example, a third alternative system includes the respiratory therapy device 110, the user interface 120, a multiple limb conduit 140, the display device 150, and the selectively adjustable underlay 43. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from the one or more sensors 210 such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

Referring to back to FIG. 1, the one or more sensors 210 include a pressure sensor 212, a flow rate sensor 214, temperature sensor 216, a motion sensor 218, a microphone 220, a speaker 222, a radio-frequency (RF) receiver 226, a RF transmitter 228, a camera 232, an infrared sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a LiDAR sensor 256, or any combination thereof. Generally, each of the one or more sensors 210 are configured to output sensor data that is received and stored in the memory device 204 or one or more other memory devices.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the infrared sensor 234, the photoplethysmogram (PPG) sensor 236, the electrocardiogram (ECG) sensor 238, the electroencephalography (EEG) sensor 240, the capacitive sensor 242, the force sensor 244, the strain gauge sensor 246, the electromyography (EMG) sensor 248, the oxygen sensor 250, the analyte sensor 252, the moisture sensor 254, and the LiDAR sensor 256, more generally, the one or more sensors 210 can include any combination and any number of each of the sensors described and/or shown herein. Any one or more of these sensors may be incorporated with the selectively adjustable underlay 43, e.g., to determine the pressure, fill rate, size, etc. of the various segments.

Referring momentarily now to FIGS. 3A-3C, a system 300 having a selectively adjustable underlay 43 is illustrated. As an option, the present system 300 may be implemented in conjunction with features from any other implementation listed herein, such as those described with reference to the other FIGS., such as FIGS. 1-2C. However, such system 300 and others presented herein may be used in various applications and/or in permutations which may or may not be specifically described in the illustrative implementations listed herein.

Looking to FIG. 3A, the selectively adjustable underlay 43 is coupled to the respiratory therapy device 110 by conduit 140. As noted above, the conduit 140 may have a variety of different configurations (e.g., single limb, multiple limb, etc.). The conduit 140 may also be coupled to the respiratory therapy device 110 and/or the inlet 330 to the adjustable underlay 43 according to any of the implementations described herein.

While any desired number and/or types of sensors may be integrated with the adjustable underlay 43, it should also be noted that sensors may also be positioned throughout the system 300 as a whole. For instance, in some implementations the respiratory therapy device 110 may include one or more sensors (e.g., see 210 of FIG. 1). These sensors may be configured to collect information pertaining to the sleep position of the user and/or their quality of sleep. According to an example, the respiratory therapy device 110 may include wave-based device, e.g., such as a sound navigation and ranging (sonar) device, a radio detection and ranging (radar) device, a LiDAR device, etc. However, any sensors that are implemented with the adjustable underlay 43 and/or the respiratory therapy device 110 may be positioned throughout the system 300. For example, a LiDAR device may be implemented in a corner of a bedroom where the adjustable underlay 43 and the respiratory therapy device 110 are positioned.

In still other implementations, data pertaining to the sleeping position of the user and/or their quality of sleep may be received from supplemental information sources that are positioned in the system. For example, a user device (e.g., 260 in FIG. 2A) may be worn by the user during the sleep session and provide positional, medical (e.g., heart rate), etc., information related to the sleeping position of the user and/or their quality of sleep during the sleep session. Similarly, devices that are positioned in a proximity to the underlay, e.g., such as an EEG sensor (e.g., see 240 of FIG. 2A), the user's cell phone, a sound machine, etc., may provide information related to the sleeping position of the user and/or their quality of sleep during the sleep session. Additional sensors (e.g., accelerometers) may be coupled to the user (e.g., fixed to clothing worn by the user, coupled to the user's skin, implanted under the user's skin, implemented in jewelry worn by the user, etc.) to provide additional positional information.

With continued reference to FIG. 3A, the inlet 330 is able to selectively distribute air provided by the respiratory therapy device 110 (or a dedicated underlay motor in other implementations) to the various segments 302, 304, 306. The inlet 330 may include any number of valves, baffles, pressure fittings, etc., that are able to selectively direct received pressurized airflow. The inlet 330 may also be coupled to any number of channels, tubing, hoses, etc., that allow for air to be provided to and/or removed from the various segments 302, 304, 306.

Uppermost segments 302 are positioned towards a “head” of the adjustable underlay 43 where a user's head is typically positioned, while middle 304 and lower 306 segments correspond to the midsection (e.g., between the user's head and hips) and legs of a user, respectively However, it should be noted that the number, shape, arrangement, etc., of the segments 302, 304, 306 is in no way intended to be limiting. It follows that the adjustable underlay 43 may have any desired configuration of segments.

The adjustable underlay 43 itself may also have different configurations depending on the implementation. For instance, while FIGS. 3A-3C depict the adjustable underlay 43 as being an adjustable sleeping pad. This adjustable underlay 43 may be separate from (e.g., not coupled to) a mattress positioned therebelow (e.g., see FIG. 2A). In other implementations the adjustable underlay may be integrated in an upper region of a mattress such that the adjustable underlay is able to adjust the sleeping surface of the mattress.

In other implementations the adjustable underlay may be configured as a selectively adjustable pillow segment, blanket, sheet, pajamas to be worn by the user, etc., or other devices that can independently support various body parts of the user. It follows that at least a portion of the adjustable underlay may be positioned between a sleeping surface (e.g., mattress, couch, floor, etc.) and the user while the user is on (e.g. using) the adjustable underlay.

With continued reference to FIG. 3A, respiratory therapy device 110 is coupled to cloud-based processor 314 and storage system 319 over network 312. Information (e.g., data, commands, metadata, requests, etc.) may be sent between the therapy device 110, the cloud-based processor 314 and the cloud-based storage system 319. According to an example, information that is collected by various sensors and components across system 300 may be sent to cloud-based storage system 319. There storage controller 316 coordinates with data storage drives 318 to store the information received from system 300. Data storage drives 318 may include any desired type of memory, e.g., such as one or more hard disk drives, solid state drives, random access memory, etc., and/or combinations thereof.

Cloud-based processor 314 may offer a scaled computing environment that is able to perform various processing operations on information stored in the cloud-based storage system 319. For example, the processor 314 may organize or generate one or more sleep-related parameters from the stored information using machine learning techniques. The one or more sleep-related parameters may include, for example, a number of adjustments made to the sleeping position of the user during a sleep session, an intensity of the adjustments made to the sleeping position of the user during the sleep session, actual sleeping positions of the user during the sleep session, an amount of user movement during the sleep session, a corresponding respiration signal, etc. These sleep-related parameters may further be correlated with the sleep that is experienced by users to understand how the sleeping position of the user during a sleep session affects the quality of sleep experienced by the user. An ideal sleeping position may thereby be determined for each user, for example, in real-time based on a number of factors related to the quality of sleep experienced by a user during a sleep session. An ideal sleeping position may also be determined based on historical information from the user or a database of users e.g. by using a machine learning algorithm.

It follows that the system 300 depicted in FIG. 3A can be used to improve the quality of sleep experienced by users by controlling the selectively adjustable underlay 43 to urge (e.g., suggest or encourage) the user to move into a sleeping position that will improve their quality of sleep. Again, the plurality of selectively adjustable segments 302, 304, 306 in the adjustable underlay 43 can each be inflated and/or deflated to achieve a desired configuration of the adjustable underlay 43. The various segments may selectively be inflated and/or deflated in order to support various parts of a user's body while sleeping. Moreover, the selectively adjustable segments may be inflated and/or deflated as desired without affecting properties of the remaining ones of the selectively adjustable segments in the adjustable underlay 43.

Looking now to FIGS. 3B-3C, cross-sectional views of the adjustable underlay 43 are shown along lines 3B-3B and 3C-3C seen in FIG. 3A according to two different implementations. As an option, either of the present cross-sectional views may be implemented in conjunction with features from any other implementation listed herein, such as those described with reference to the other FIGS., such as FIGS. 1-3A. However, such cross-sectional views in FIGS. 3B-3C and others presented herein may be used in various applications and/or in permutations which may or may not be specifically described in the illustrative implementations listed herein. It should also be noted that the various dimensions, spacing, general shapes, etc., of the various components in FIGS. 3B-3C may be exaggerated for illustrative purposes which is in no way intended to limit the invention. For instance, the size, shape, orientation, number, etc., of the various segments 304, 302 may vary depending on the particular implementation.

Looking first to FIG. 3B, the various segments 304 are shown as being filled to different levels. Specifically, in the present implementation the leftmost segment 304 is filled to a maximum thickness t₁, while the remaining segments 304 are filled progressively less moving towards the center of the adjustable underlay 43, such that t₂ is less than t₁, t₃ is less than t₂, and t₄ is less than t₃. As a result, the overall thickness of the adjustable underlay 43 is also affected such that the outer edge of the adjustable underlay 43 has a greater thickness ta than a thickness tb towards a middle region of the adjustable underlay 43. This varying thickness of the adjustable underlay 43 may cause or urge a user to turn onto their side during a sleep session in order to improve the quality of sleep experienced by the user.

For example, a user that is identified as sleeping on their back using one or more pressure sensors 320, 321, 322, audio signals recorded by a microphone to determine sleeping patterns, positional information received from one or more inertial sensors, (e.g., accelerometers, gyroscopes, magnetometers, etc.), etc. may be urged to roll onto their side as a result of adjusting the amount of air included in each of the segments 304 as shown in FIG. 3B. Additional adjustments may be made to the various segments 304 based on how successful initial settings are at changing the sleeping position of the user and/or improving the quality of sleep experienced by the user. For instance, in some situations changes to the segments 304 may be exaggerated in response to determining the user has not been sufficiently urged to shift their actual sleeping position to an intended sleeping position. In other situations, the segments 304 may be changed to a different configuration altogether in response to determining that the quality of sleep experienced by the user has not been improved, despite causing the user to shift their actual sleeping position to an intended sleeping position.

As noted above, processor 202 and/or memory device 204 can receive data (e.g., physiological data, audio data, segment configuration data, etc.) from the one or more sensors. Accordingly, sensors 320, 321, 322 in some implementations are able to communicate with the processor 202 and/or memory device 204 directly, e.g., over a wired and/or wireless connection. In other implementations, sensors 320, 321, 322 integrated with the adjustable underlay 43 may communicate with one or more intermediary components. For instance, the sensors 320, 321, 322 may communicate with a control module (not shown) of the adjustable underlay 43 that is able to communicate with the processor 202 and/or memory device 204 directly, e.g., over a wired and/or wireless connection. It follows that the sensors 320, 321, 322 and/or the adjustable underlay 43 itself may include an antenna, a receiver (e.g., an RF receiver), a transmitter (e.g., an RF transmitter), a transceiver, or any combination thereof. Such components can be coupled to or integrated in the sensors 320, 321, 322 and/or an outer periphery of the adjustable underlay 43 itself.

While sensors 320, 321, 322 are described herein as including pressure sensors, they may include any desired type of sensor. For instance, in some implementations one or more of sensors 320, 321, 322 include pressure sensors integrated with the adjustable underlay, while one or more other ones of the sensors 320, 321, 322 include pneumatic sensors integrated with the adjustable underlay 43. It follows that any desired type, orientation, configuration, placement, etc., of sensors may be integrated with the adjustable underlay 43 in order to collect desired information (e.g., positional information) related to a user's sleep session. In still other implementations, ones of the sensors 320, 321, 322 may include flow sensors capable of recording the amount of air, liquid, etc., that is provided to and/or removed from the various segments.

While some segments may be used to urge the user to adjust their sleeping position, other segments may be used to maintain a desirable sleeping environment. For example, while segments 304 may be adjusted to urge a user to roll on their side, looking to FIG. 3C, segments 302 may remain at a predetermined setting to achieve preferred neck alignment of the user regardless of their sleeping position. The thickness ts of segments 302 may correspond to physiological details of the user, preferences set by the user, sleep-related parameters organized and/or generated by machine learning techniques, corresponding sleep quality, etc. This thickness ts of segments 302 causes the head region of the adjustable underlay 43 to have an overall thickness te that is less than a maximum achievable thickness tmax.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

In some implementations, operation of the selectively adjustable underlay 43 may be considered when generating the one or more sleep-related parameters. The one or more sleep-related parameters that can be determined for the user 20 during the sleep session may thereby include, for example, a number of adjustments made to the sleeping position of the user during a sleep session, an intensity of the adjustments made to the sleeping position of the user during the sleep session, an amount of user movement during the sleep session, a respiration signal, etc.

The one or more sensors 210 can be used to generate, for example, physiological data, audio data, or both. Physiological data generated by one or more of the sensors 210 can be used by the control system 200 to determine a sleep-wake signal associated with the user 20 (FIG. 2A) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopneas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof. In other implementations, the pressure sensors 212 are piezoelectric sensors that generate sensor data indicative of the amount of pressure in each segment of the adjustable underlay 43.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20 (FIG. 2A), a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones

The speaker 222 outputs sound waves that are audible to a user of the system 10 (e.g., the user 20 of FIG. 2A). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260.

The microphone 220 and the speaker 222 can be used as separate devices. In some implementations, the microphone 220 and the speaker 222 can be combined into an acoustic sensor 224 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human ear (e.g., below 20 Hz or above around 18 kHz) so as not to disturb the sleep of the user 20 or the bed partner 30 (FIG. 2A). Based at least in part on the data from the microphone 220 and/or the speaker 222, the control system 200 can determine a location of the user 20 (FIG. 2A) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g. a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user (FIG. 2A), to determine air flow of the mouth and/or nose of the user, to determine a time when the user enters the bed (FIG. 2A), and to determine a time when the user exits the bed. In some implementations, the camera 232 includes a wide angle lens or a fish eye lens.

The infrared (IR) sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 (FIG. 2A) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120 and/or the associated headgear (e.g., straps, etc.).

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 174 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 174 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The Light Detection and Ranging (LiDAR) sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 100, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 132 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 (FIG. 1) includes a display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 100 also includes an activity tracker 270. The activity tracker 270 is generally used to aid in generating physiological data associated with the user. The activity tracker 270 can include one or more of the sensors 210 described herein, such as, for example, the motion sensor 138 (e.g., one or more accelerometers and/or gyroscopes), the PPG sensor 154, and/or the ECG sensor 156. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2A, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 100 also includes a blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2A, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra wideband ranges (which may include spreading, PRN codes or impulse systems).

While the control system 200 and the memory device 204 are described and shown in FIG. 1 as being a separate and distinct component of the system 100, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user 20 has laid or sat down in the bed 40 (or another area or object on which they intend to sleep), and has turned on the respiratory therapy device 110 and donned the user interface 120. The sleep session can thus include time periods (i) when the user 20 is using the respiratory therapy system 100, but before the user 20 attempts to fall asleep (for example when the user 20 lays in the bed 40 reading a book); (ii) when the user 20 begins trying to fall asleep but is still awake; (iii) when the user 20 is in a light sleep (also referred to as stage 1 and stage 2 of non-rapid eye movement (NREM) sleep); (iv) when the user 20 is in a deep sleep (also referred to as slow-wave sleep, SWS, or stage 3 of NREM sleep); (v) when the user 20 is in rapid eye movement (REM) sleep; (vi) when the user 20 is periodically awake between light sleep, deep sleep, or REM sleep; or (vii) when the user 20 wakes up and does not fall back asleep.

The sleep session is generally defined as ending once the user 20 removes the user interface 120, turns off the respiratory therapy device 110, and gets out of bed 40. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user 20, ending when the respiratory therapy device 110 stops supplying the pressurized air to the airway of the user 20, and including some or all of the time points in between, when the user 20 is asleep or awake.

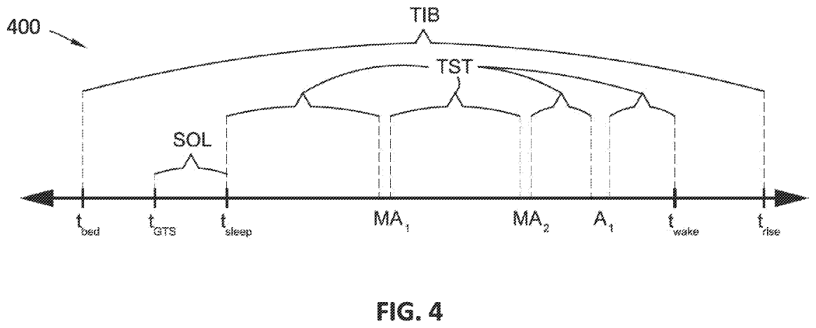

Referring to the timeline 400 in FIG. 4 the enter bed time t_{bed} is associated with the time that the user initially enters the bed (e.g., bed 40 in FIG. 2A) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (tGTs) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 400 of FIG. 4, the total sleep time (TST) spans between the initial sleep time tₛₗₑₑₚ and the wake-up time twakc, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (tbcd) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (tGrs) and ending at the wake-up time (twakc). In some implementations, a sleep session is defined as starting at the go-to-sleep time (tGTs) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

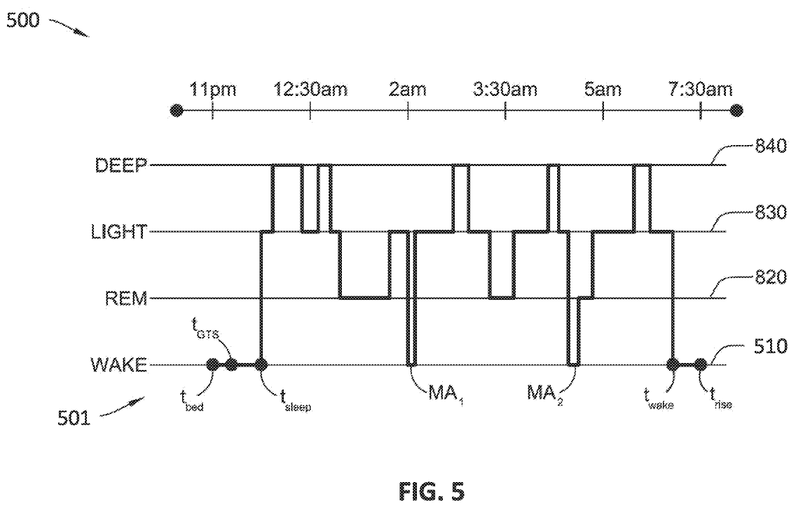

Referring to FIG. 5, an exemplary hypnogram 500 corresponding to the timeline 400 (FIG. 4), according to some implementations, is illustrated. As shown, the hypnogram 500includes a sleep-wake signal 501, a wakefulness stage axis 510, a REM stage axis 520, a light sleep stage axis 530, and a deep sleep stage axis 540. The intersection between the sleep-wake signal 501 and one of the axes 510-540 is indicative of the sleep stage at any given time during the sleep session.

The sleep-wake signal 501 can be generated based on physiological data associated with the user (e.g., generated by one or more of the sensors 210 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 500 is shown in FIG. 5 as including the light sleep stage axis 530 and the deep sleep stage axis 540, in some implementations, the hypnogram 500can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 204.

The hypnogram 500 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (tGrs) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (tbcd), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (tbcd), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 218, the microphone 220, the camera 232, or any combination thereof. The go-to-sleep time can be determined based on, for example, data from the motion sensor 218 (e.g., data indicative of no movement by the user), data from the camera 232 (e.g., data indicative of no movement by the user and/or that the user has turned off the lights) data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

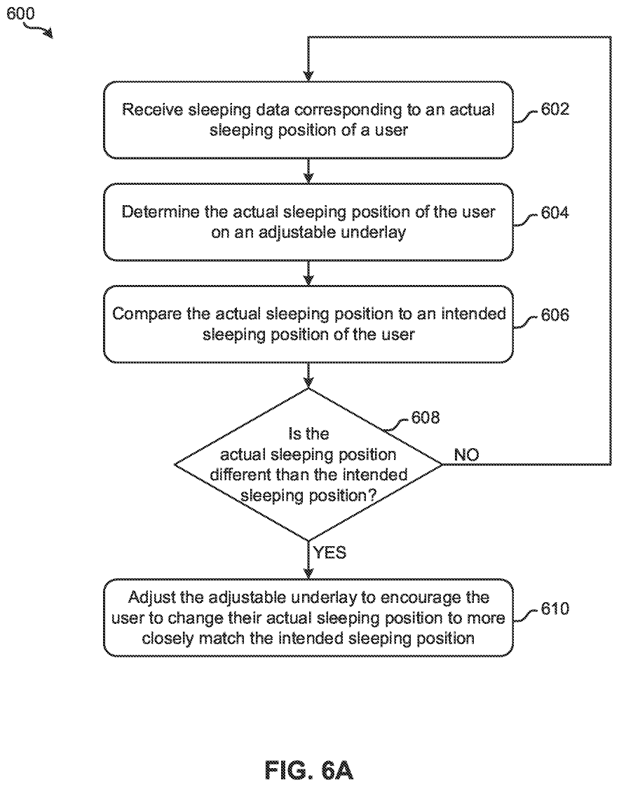

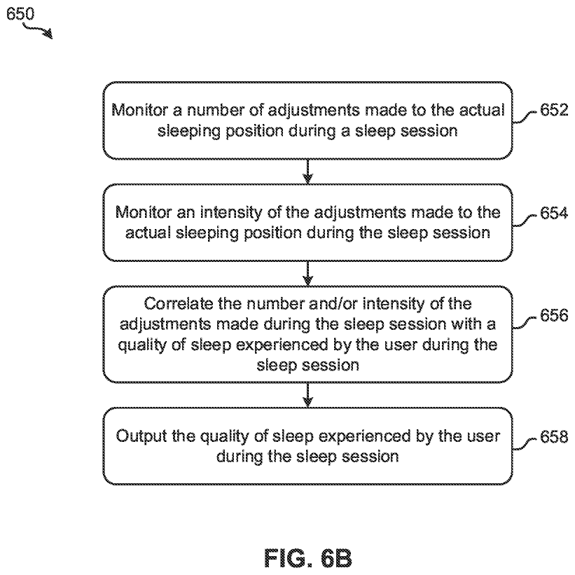

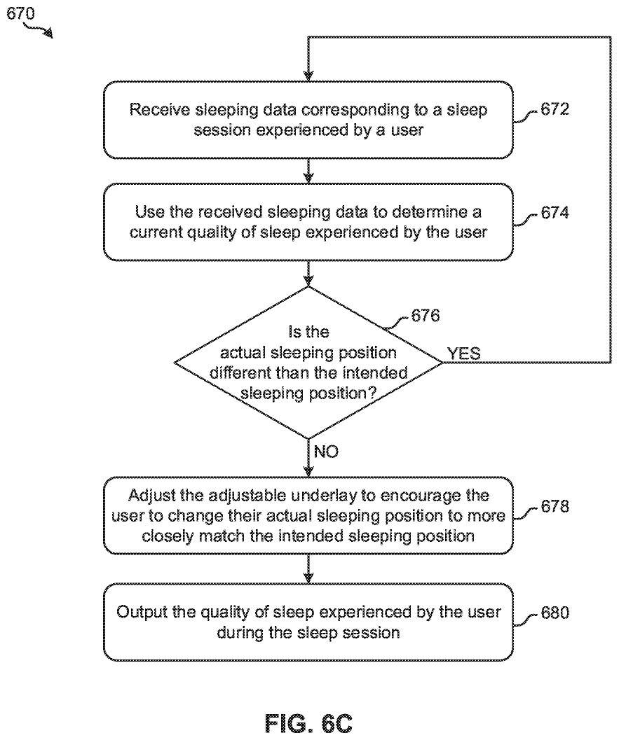

Referring generally to FIG. 6A, a method 600 for improving the quality of sleep experienced by a user by selectively adjusting the sleeping position of the user is illustrated according to some implementations of the present disclosure. People often have a difficult time finding and staying in a comfortable sleeping position. For example, pregnant women and people with chronic back pain suffer from poor sleep because they have difficulty finding, and staying in, a comfortable sleeping position. Additionally, many people struggle from disabilities, and stiffness, chronic back pain, age, etc., and others prefer sleeping in a certain position. It follows that these user's quality of sleep may be improved by providing an adjustable bed underlay with independently inflatable and deflatable segments that adjust to support various parts of a user's body while sleeping. As noted above, the adjustable underlay may be positioned between a sleeping surface (e.g., a mattress) and the user, and can be in the form of a sheet, blanket, pillow, etc., that is able to independently support various body parts of the user while asleep.

One or more steps of the method 600 can be implemented using any element or aspect of the systems 100, 300 (FIGS. 1-3C) described herein. It should also be noted that more or less operations than those specifically described in FIG. 6A may be included, as would be understood by one of skill in the art upon reading the present descriptions.

Each of the steps of the method 600 may be performed by any suitable component of the operating environment. For example, in various implementations, the method 600 may be partially or entirely performed by a controller, a processor, a computer, etc., or some other device having one or more processors therein. Thus, in some implementations, method 600 may be a computer-implemented method. Other implementations include computer program product having instructions which, when executed by a computer, cause the computer to carry out the method 600. The computer program product may even be a non-transitory computer readable medium. Moreover, the terms computer, processor and controller may be used interchangeably with regards to any of the implementations herein, such components being considered equivalents in the many various permutations of the present invention.

Moreover, for those embodiments having a processor, the processor, e.g., processing circuit(s), chip(s), and/or module(s) implemented in hardware and/or software, and preferably having at least one hardware component may be utilized in any device to perform one or more steps of the method 600. Illustrative processors include, but are not limited to, a central processing unit (CPU), an application specific integrated circuit (ASIC), a field programmable gate array (FPGA), etc., combinations thereof, or any other suitable computing device known in the art.

As shown in FIG. 6A, operation 602 of method 600 includes receiving sleeping data corresponding to an actual sleeping position of a user. The sleeping data may be received from one or more sensors integrated with an adjustable underlay, from the user via a GUI, from one or more sensors in the vicinity of the user during a sleep session (e.g., cameras, microphones, a sound navigation and ranging (sonar) device, a radio detection and ranging (radar) device, and a LiDAR device, etc.), etc. It follows that the type of sleeping data received in operation 602 may also vary depending on the implementation. For instance, the sleeping data may include audio recordings, video recordings, wave-based positional information, movement-based positional information, pressure-based positional information, etc. The received sleeping data may also include information corresponding to the quality of sleep experienced by the user (e.g., audio clips recorded by a microphone to identify breath and/or sleep quality of the user).

In some implementations, the sleeping data received indicates a pre-set sleeping position that identifying various settings for the plurality of selectively adjustable segments. The pre-set sleeping position may be selected by the user to control their sleeping position based on a number of factors. For instance, a particular sleeping position may be recommended and set based on medical conditions experienced by the user, including but not limited to, pregnancy, back ache, neck ache, reflux and/or heartburn, allergies, snoring, OSA, hypertension, etc.

Operation 604 includes determining the actual sleeping position of the user on an adjustable underlay. The actual sleeping position of the user may be determined based at least in part on the received sleeping data. Additional information, including information received during past sleep sessions, correlations determined between different data using machine learning techniques, sleep quality data, etc., may also be considered to determine the actual sleeping position of the user in some implementations.

According to some implementations, positional-based information may be used to determine the actual sleeping position of the user. For example, pressure readings from sensors integrated with the adjustable underlay may be evaluated to determine a general position of the user on the underlay. This general position may further be improved by incorporating data captured by one or more wave-based devices, e.g., such as a sonar device, a radar device, a LiDAR device, etc. Operation 604 may thereby be able to start with an approximation of the user's actual sleeping position, and improve the accuracy of the sleeping position by evaluating additional information. In some implementations, the actual position of the user may be evaluated (e.g., improved upon) until a predetermined confidence score may be assigned to the approximation.

With continued reference to FIG. 6A, operation 606 includes comparing the actual sleeping position to an intended sleeping position of the user. The results of this comparison are further used to determine if the actual sleeping position is different than the intended sleeping position. See decision 608.

Different implementations may achieve this comparison and/or determination in a variety of ways. For instance, differences between the current and ideal sleeping positions may be quantified and compared to a predetermined range that may define an acceptable (e.g., preferred) level of similarity between the two positions. These differences may be quantified and/or compared using any desired machine learning techniques. Moreover, the predetermined ranges may be set by the user(s), based on sleep quality experienced during past sleep sessions, adjusted dynamically in response to information received that is associated with the current sleep session, etc.

As a result, processes 606, 608 are desirably able to monitor the current sleep position of a user and determine whether it should be adjusted to better match an intended sleep position that may be configured to improve the quality of sleep experienced by the user. It should also be noted that while the quality of sleep experienced by the user may be able to quantify the sleep improvements achieved by adjusting the user's sleep position during a sleep cycle, it may also be evaluated in determining whether the underlay should be adjusted to urge the user to move from their current sleeping position (e.g., see FIG. 6C below).

In some implementations, comparing the actual sleeping position to an intended sleeping position of the user includes determining an amount of time the intended sleeping position has been implemented. While a particular sleeping position may be beneficial for a user, it may not be desirable that the user remains in that sleeping position for the entire sleep session. This may be based on user preference, medical conditions of the user, determined sleeping habits of the user, inputs from medical professionals, etc. The quality of sleep experienced by the user may thereby be improved by shifting between ideal sleeping positions based on how long the user has been in their actual sleeping position.

In response to determining that the intended sleeping position has been used to adjust the actual sleeping position for a predetermined amount of time, an update to the intended sleeping is preferably made. The adjustable underlay may thereby be modified to encourage the user to change the actual sleeping position to the updated intended sleeping position. This update thereby allows for the user to be shifted to (e.g., urged towards) a different intended sleeping position. As a result, the overall quality of sleep experienced by the user during the sleep session is improved. In some instances, changes to the intended sleeping position may be based on ergonomic data which is used to determine sleeping positions that are best suited for the user's specific dimensions and preferences. The underlay may even be able to perform actions that increase the likelihood that the user will move into the updated intended sleeping position. For example, the underlay may periodically vibrate with increasing intensity until movement of the user towards the intended sleeping position is registered.

In response to determining that the actual sleeping position is not different than (e.g., is sufficiently similar to) the intended sleeping position, method returns to operation 602 from decision 608. Accordingly, any one or more of the processes included in method 600 may be repeated in order to process additional sleeping data. In other words, any one or more of the processes included in method 600 may be repeated for subsequently received sleeping data such that the user's sleep position is monitored throughout the sleep session and a quality of sleep experienced by the user is optimized. Some or all of the processes included in method 600 may thereby be performed in the background compared to other active sleep routines. For instance, inflating and/or deflating the various segments of the adjustable underlay may be performed in parallel with, and while considering, the pressurized air being provided to a user interface (e.g., 120 of FIGS. 1-2A). It follows that various ones of the processes included in method 600 may be repeated in an iterative fashion during a sleep session.

Returning to decision 608, method 600 proceeds to operation 610 in response to determining that the actual sleeping position is different than the intended sleeping position. There, operation 610 includes adjusting the adjustable underlay to encourage the user to change their actual sleeping position to more closely match the intended sleeping position.

In some implementations, operation 610 may include sending one or more instructions, commands, requests, etc., to a motor which is able to actually inflate and/or deflate the various segments of the adjustable underlay. According to an example, which is in no way intended to limit the invention, instructions may be sent from cloud-based processor 314 and/or storage system 319 of FIG. 3A over network 312 to cause one or more motors in the respiratory therapy device 110 to produce the desired airflows. In other implementations, operation 610 can be performed by control system 200 of FIG. 1, such that adjustments to the underlay are made by directly sending instructions to the motor and/or any valves in the inflatable bed underlay to operate in a desired manner.

As noted above, the selectively adjustable segments may be inflated and/or deflated without affecting the amount that remaining ones of the segments are currently inflated. This may be achieved by using an inlet (e.g., see 330 of FIG. 3A) that is able to selectively distribute air to the various segments. The inlet may include any number of one-way valves, baffles, pressure fittings, etc., that are able to selectively direct received pressurized airflow. The inlet may also be coupled to any number of channels, tubing, hoses, etc., that allow for air to be provided to and/or removed from the various segments.

While various ones of the implementations herein have been described in the context of using air to inflate and/or deflate the segments of the adjustable underlay, it should again be noted that any desired type of material may be used to adjust the size, firmness, orientation, etc., of the various segments. For instance, adjusting the adjustable underlay includes inflating and/or deflating the adjustable underlay with one or more of gasses (e.g., ambient air), liquids (e.g., water), other materials, etc., that would be apparent to one skilled in the art after reading the present description.

Looking now to FIG. 6B, a method 650 for monitoring the adjustments made to the underlay is illustrated according to some implementations of the present disclosure. One or more steps of the method 650 can be implemented using any element or aspect of the system 100 (FIGS. 1-6A) described herein. For instance, method 650 may be performed in the background such that it does not affect operation of the adjustable underlay during a sleep session, e.g., as would be appreciated by one skilled in the art after reading the present description. It should also be noted that more or less operations than those specifically described in FIG. 6A may be included, as would be understood by one of skill in the art upon reading the present descriptions

As shown, operation 652 of method 650 includes monitoring a number of adjustments made to the actual sleeping position during a sleep session, while operation 654 includes monitoring an intensity of the adjustments made to the actual sleeping position during the sleep session. With respect to the present description, the “intensity” of the adjustments made refers to the overall amount by which the various segments are inflated and/or deflated. For example, a segment that is inflated from 5% to 90% of total capacity will have experienced a more intense adjustment than a different segment that is inflated from 50% to 55% of total capacity.

The number and intensity of the adjustments that are made to the actual sleeping position of a user during a sleep session has an effect on the overall quality of sleep experienced by the user. Accordingly, by monitoring these adjustments that are made, method 650 is desirably able to correlate a user's sleep quality with information associated with the user's position during a sleep session. Operation 656 thereby includes correlating the number and/or intensity of the adjustments made during the sleep session with a quality of sleep experienced by the user during the sleep session. This correlation between the adjustments made to the underlay and the quality of sleep experienced by the user may be made using any one or more of the processes described herein.

For example, a processor may organize or generate one or more sleep-related parameters using machine learning techniques. The one or more sleep-related parameters may include, for example, a number of adjustments made to the sleeping position of the user during a sleep session, an intensity of the adjustments made to the sleeping position of the user during the sleep session, actual sleeping positions of the user during the sleep session, an amount of user movement during the sleep session, a corresponding respiration signal, etc. These sleep-related parameters may further be correlated with the sleep that is experienced by users to understand how the sleeping position of the user during a sleep session affects the quality of sleep experienced by the user. An ideal sleeping position may thereby be determined for each user in real-time based on a number of factors related to the quality of sleep experienced by a user during a sleep session.

Proceeding to operation 658, the quality of sleep experienced by the user during the sleep session is output. The quality of sleep may be output to a display device of a respiratory therapy system (e.g., see 150 of system 100 in FIG. 1). The user may thereby be able to see the quality of sleep they experienced during their past sleep sessions. The information output in operation 658 may also include data that indicates any improvements to the quality of sleep experienced by the user as a result of the adjustable underlay being used to adjust their sleeping position during the sleep session. This may be compared to a predicted quality of sleep that would have been experienced by the user had their sleeping position not been adjusted during the sleep session. In still further implementations, the information output in operation 658 may suggest one or more changes to the intended sleeping position of the user during subsequent sleep sessions. These changes are preferably configured to improve the quality of sleep experienced by the user during the subsequent sleep sessions.

In some implementations, the quality of sleep experienced by the user may determine whether adjustments are made to the underlay. Referring now to FIG. 6C, a method 670 for improving the quality of sleep experienced by a user by selectively adjusting the sleeping position of the user based on a quality of sleep experienced by the user is illustrated according to some implementations of the present disclosure. One or more steps of the method 670 can be implemented using any element or aspect of the systems 100, 300 (FIGS. 1-3C) described herein. It should also be noted that more or less operations than those specifically described in FIG. 6C may be included, as would be understood by one of skill in the art upon reading the present descriptions.

Operation 672 includes receiving sleeping data corresponding to a sleep session experienced by a user. The sleeping data may be received from one or more sensors integrated with an adjustable underlay, from the user via a GUI, from one or more sensors in the vicinity of the user during a sleep session (e.g., cameras, microphones, a sound navigation and ranging (sonar) device, a radio detection and ranging (radar) device, and a LiDAR device, etc.), etc. It follows that the type of sleeping data received in operation 672 may also vary depending on the implementation. For instance, the received sleeping data preferably include information corresponding to the quality of sleep experienced by the user (e.g., audio clips recorded by a microphone to identify breath and/or sleep quality of the user). Accordingly, the sleeping data may include audio recordings, video recordings, wave-based positional information, movement-based positional information, pressure-based positional information, etc.

Operation 674 further includes using the received sleeping data to determine a current quality of sleep experienced by the user. As noted above, in some implementations the number and intensity of the adjustments that are made to the underlay during a sleep session has an effect on the overall quality of sleep experienced by the user. Accordingly, by monitoring these adjustments that are made, method 670 is desirably able to correlate a user's sleep quality with information associated with the user's position during a sleep session. Operation 674 thereby includes correlating the number and/or intensity of the adjustments made during the sleep session with a quality of sleep experienced by the user during the sleep session. This correlation between the adjustments made to the underlay and the quality of sleep experienced by the user may be made using any one or more of the processes described herein.

Additional details included in the received sleeping data may also be used to determine the current quality of sleep experienced by the user. For example, the current quality of sleep experienced by the user may be impacted by quality of sleep experienced by the user during a previous sleep session, earlier in the same sleep session, etc. The ambient temperature in the sleeping environment, the presence of external noises, unprompted movement by the user during the sleep session, etc., may also be evaluated to determine a current quality of sleep experienced by the user.

Decision 676 includes determining if the current quality of sleep is in a predetermined range. The range may be set by the user, determined based on the received sleeping data, be dynamically adjusted based on past quality of sleep, etc. It should also be noted that “in a predetermined range” is in no way intended to limit the invention. Rather than determining whether a value is in in a predetermined range, equivalent determinations may be made, e.g., as to whether a value is above a threshold, whether a value is outside a predetermined range, whether an absolute value is above a threshold, whether a value is below a threshold, etc., depending on the desired implementation.

In response to determining that the current quality of sleep is in the predetermined range, method returns to operation 672 from decision 676. Method 670 thereby includes intentionally refraining from adjusting the adjustable underlay for at least a portion of time. In some implementations method 670 may include waiting for a predetermined amount of time to pass or some other condition to be satisfied before any further adjustments are made to the underlay.

Upon returning to operation 672, any one or more of the processes included in method 670 may be repeated in order to process additional sleeping data. In other words, any one or more of the processes included in method 670 may be repeated for subsequently received sleeping data such that the user's quality of sleep is monitored throughout the sleep session and improved by selectively adjusting the underlay. Some or all of the processes included in method 670 may thereby be performed in the background compared to other active sleep routines. For instance, inflating and/or deflating the various segments of the adjustable underlay may be performed in parallel with, and while considering, the pressurized air being provided to a user interface (e.g., 120 of FIGS. 1-2A). It follows that various ones of the processes included in method 670 may be repeated in an iterative fashion during a sleep session.

Returning to decision 676, method 670 proceeds to operation 678 in response to determining that the current quality of sleep is not in the predetermined range. There, operation 678 includes adjusting the adjustable underlay to encourage the user to change their actual sleeping position to more closely match the intended sleeping position. The underlay may be adjusted using any one or more of the processes and/or implementations described herein.

From operation 678, method 670 proceeds to operation 680 where the quality of sleep experienced by the user during the sleep session is output. As noted above, the quality of sleep may be output to a display device of a respiratory therapy system (e.g., see 150 of system 100 in FIG. 1). The user may thereby be able to see the quality of sleep they experienced during their past sleep sessions. The information output in operation 680 may also include data that indicates any improvements to the quality of sleep experienced by the user as a result of the adjustable underlay being used to adjust their sleeping position during the sleep session. This may be compared to a predicted quality of sleep that would have been experienced by the user had their sleeping position not been adjusted during the sleep session. In still further implementations, the information output in operation 680 may suggest one or more changes to the intended sleeping position of the user during subsequent sleep sessions. These changes are preferably configured to improve the quality of sleep experienced by the user during the subsequent sleep sessions.

While various ones of the implementations included herein have described the adjustable underlay as being modified during a sleep session in response to various information collected during the sleep session, in other implementations the adjustable underlay may rely on user selection and/or manual operation of the various segments in the underlay. Accordingly, the user may fill the segments of the underlay to a desired configuration before the start of a sleep session, whereby the segments are not adjusted during the sleep session.

In other implementations, two or more underlays may be used in combination with each other. Two users lying on a same sleep surface may thereby be positioned by the different underlays such that each experiences an improved quality of sleep despite differences in the ideal sleeping positions of the users. For example, one user may be pregnant and would experience improved sleep quality by being positioned on their side in a way that accommodates their changing anatomy, while the other user may suffer from snoring and would experience improved sleep quality by being positioned on their stomach.

One or more elements or aspects or steps, or any portion(s) thereof, from one or more of any of claims 1 to 26 below can be combined with one or more elements or aspects or steps, or any portion(s) thereof, from one or more of any of the other claims 1 to 26 or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

1. A method, comprising:
receiving sleeping data corresponding to an actual sleeping position of a user;
determining the actual sleeping position on an adjustable underlay, the adjustable underlay having a plurality of selectively adjustable segments;
comparing the actual sleeping position to an intended sleeping position of the user; and
in response to determining that the actual sleeping position is different than the intended sleeping position, adjusting the adjustable underlay to encourage the user to change the actual sleeping position to the intended sleeping position.

2. The method of claim 1, wherein adjusting the adjustable underlay includes adjusting one or more of the plurality of selectively adjustable segments without affecting remaining ones of the plurality of selectively adjustable segments.

3. The method of claim 1, wherein adjusting the adjustable underlay includes inflating and/or deflating the adjustable underlay with one or more of air, a liquid, or other materials.

4. The method of claim 1, wherein the adjustable underlay is inflatable with air, the plurality of selectively adjustable segments being individually inflatable with air such that inflation and/or deflation of any segment leaves any other segment unaffected.

5. The method of claim 1, further comprising:
monitoring a number of adjustments made to the actual sleeping position during a sleep session;
correlating the number of adjustments made during the sleep session with a quality of sleep experienced by the user during the sleep session; and
outputting the quality of sleep experienced by the user during the sleep session.

6. The method of claim 1, further comprising:
monitoring an intensity of the adjustments made to the actual sleeping position during the sleep session; and
correlating the intensity of the adjustments made and the number of adjustments made during the sleep session with the quality of sleep experienced by the user during the sleep session.

7. The method of claim 1, wherein outputting the quality of sleep experienced by the user during the sleep session includes:
suggesting one or more changes to the intended sleeping position, the changes being configured to improve the quality of sleep experienced by the user during a subsequent sleep session.

8. The method of claim 1, wherein at least a portion of the adjustable underlay is positioned between a sleeping surface and the user while the user is on the adjustable underlay.

9. The method of claim 1, wherein at least one of the plurality of selectively adjustable segments is a pillow segment, wherein the intended sleeping position includes a neck alignment of the user.

10. The method of claim 1, wherein the sleeping data corresponding to the actual sleeping position of the user is received from one or more accelerometers coupled to the user and/or positioned in a vicinity of the user.

11. The method of claim 1, wherein the sleeping data corresponding to the actual sleeping position of the user is received from one or more pressure and/or pneumatic sensors integrated with the adjustable underlay.

12. The method of claim 1, wherein the sleeping data corresponding to the actual sleeping position of the user is wave-based data received from a device selected from the group consisting of a sound navigation and ranging (sonar) device, a radio detection and ranging (radar) device, and a light detection and ranging (LiDAR) device.

13. The method of claim 1, further comprising:
using the received sleeping data to determine a current quality of sleep experienced by the user;
in response to determining that the current quality of sleep is not in a predetermined range, adjusting the adjustable underlay to encourage the user to change the actual sleeping position to the intended sleeping position.

14. (canceled)

15. The method of claim 1, wherein the adjustable underlay is separate from a mattress.

16. The method of claim 1, wherein the adjustable underlay is integrated in an upper region of a mattress.

17. The method of claim 1, wherein comparing the actual sleeping position to an intended sleeping position of the user includes:
updating the intended sleeping position in response to determining that the intended sleeping position has been used to adjust the actual sleeping position for a predetermined amount of time; and
in response to determining that the actual sleeping position is different than the updated intended sleeping position, adjusting the adjustable underlay to encourage the user to change the actual sleeping position to the updated intended sleeping position.

18. The method of claim 1, wherein comparing the actual sleeping position to an intended sleeping position of the user includes:
in response to determining that a quality of sleep experienced by the user is outside a predetermined range, adjusting the adjustable underlay to encourage the user to change the actual sleeping position to a sleeping position configured to improve the quality of sleep experienced by the user; and
outputting the quality of sleep experienced by the user during the sleep session.

19. (canceled)

20. The method of claim 1, further comprising:
receiving a selection indicating a pre-set sleeping position, the pre-set sleeping position identifying settings for the plurality of selectively adjustable segments; and
identifying the pre-set sleeping position as the intended sleeping position.

21-24. (canceled)

25. A system comprising:
an adjustable underlay having a plurality of selectively adjustable segments;
a memory device having stored thereon machine-readable instructions; and
a control system including one or more processors configured to execute the machine-readable instructions to:
receive sleeping data corresponding to an actual sleeping position of a user;
determine the actual sleeping position on an adjustable underlay, the adjustable underlay having a plurality of selectively adjustable segments;
compare the actual sleeping position to an intended sleeping position of the user; and
in response to determining that the actual sleeping position is different than the intended sleeping position, adjust the adjustable underlay to encourage the user to change the actual sleeping position to the intended sleeping position.

26. A system comprising:
an adjustable underlay having a plurality of selectively adjustable segments, each of which may selectively be inflated or deflated to adjust the amount of air included in the respective segment;
a respiratory therapy system configured to selectively supply pressurized air to a user and/or the adjustable underlay;
a memory device having stored thereon machine-readable instructions; and
a control system including one or more processors configured to execute the machine-readable instructions to:
receive sleeping data corresponding to an actual sleeping position of the user;
determine the actual sleeping position on an adjustable underlay, the adjustable underlay having a plurality of selectively adjustable segments;
compare the actual sleeping position to an intended sleeping position of the user; and
in response to determining that the actual sleeping position is different than the intended sleeping position, adjust the adjustable underlay to encourage the user to change the actual sleeping position to the intended sleeping position.
